(19) United States
(12) Patent Application Publication
(10) Pub. No.: US 2005/0071895 A1
(43) Pub. Date: Mar. 31, 2005

(54) Generation of transgenic mice by transgene-mediated rescue of spermatogenesis

(76) Inventor: Nian Zhang, Grand Rapids, MI (US)

Correspondence Address: VENABLE, BAETJER, HOWARD AND CIVILETTI, LLP, P.O. BOX 34385, WASHINGTON, DC, 20043-9998, US

(21) Appl. No.: 10/498,113

(22) PCT Filed: Dec. 11, 2002
(86) PCT No.: PCT/US02/39525

Related U.S. Application Data
(60) Provisional application No. 60/338,659, filed on Dec. 11, 2001.

(51) Int. Cl. A01K 67/027; C12N 15/85

(52) U.S. Cl. 800/18; 435/455; 435/320.1

(57) ABSTRACT

The present invention provides transgenic mice and nucleic acid constructs and methods for creating them. The transgenic mice are generated preferably by electroporation-mediated transfection of a nucleic acid construct into the testes of a sterile spermatogenesis-deficient male mouse. The construct includes DNA encoding a spermatogenesis rescue factor resulting in rescue of spermatogenesis while contemporaneously transferring a nucleic acid of interest for expression in the transgenic mouse so that progeny sired by the transgenic mouse also harbor the transgene. Certain embodiments of the invention also utilize (a) the salmonid derived Sleeping Beauty transposition system to augment genomic integration of the transgene and/or (b) the Cre/loxP recombination system for excision from sired progeny of the DNA responsible for spermatogenesis rescue.

### BACKGROUND OF THE INVENTION

1. Field of the Invention

The invention in the field of molecular and developmental biology relates to a novel method for generating transgenic mammals, genetic constructs useful therein, and novel genetically modified mammals.

2. Description of the Background Art

In the most widely used method for producing transgenic animals, DNA is microinjected into the pronuclei of one-cell stage embryos (Gordern et al 1980. Proc. Natl. Acad. Sci. USA 77:7380-7384. The resulting animals are propagated, transmitting the transgene in the germline (Brinster et al. (1981) Cell 27:223-231; Costantini and Lacy(1981), Nature 294:92-94; Gorden and Ruddle (1991) Science 214:1244-1246; E. Wagner et al. (1981) Proc. Natl. Acad. Sci. USA 78:5016-5020; T. Wagner et al.(1981) Proc. Natl. Acad. Sci. USA 78:6376-6380). Wagner and Hoppe, U.S. Pat. No. 4,873,191, disclose genetic transformation of a zygote (typically pronuclei of a male zygote) by microinjection of exogenous genetic material into the nucleus which ultimately forms at least a part of the nucleus of the zygote. The zygote is then allowed to undergo differentiation and development into the organism. The genotype of the zygote, and the resulting organism, includes the gene(s) present of the exogenous genetic material which is phenotypically expressed.

In spite of its widespread use, the method described above is tedious. Generally, three to four hundred eggs are needed for microinjection, requiring collection from at least ten superovulated female mice who receive hormone injections one day before egg collection after priming hormonally (e.g., with pregnant mare serum) two days before mating. These females are mated to males, and fertilized eggs are collected the following morning. DNA constructs are injected into male pronuclei with the aid of a specialized microscope after which the eggs are surgically implanted into the oviducts of pseudopregnant females that had been mated to vasectomized males the day before ((Hogan et al. (1994) Manipulating the Mouse Embryo: A Laboratory Manual, 2^{nd} Ed., Cold Spring Harbor Laboratory Press, Cold Spring Harbor, N.Y., pp. 218-252).

Other methods of introducing exogenous DNA into embryos rely on viral infection. Three major approaches have been employed. One group of methods relies on infection of early mouse embryos by co-culture with retrovirus-producing cells (Hogan et al., supra, pp. 251-252). Alternative methods use infection of either pre- or post-implantation mouse embryos with wild-type or recombinant retroviruses (Jaenisch et al. (1976) Proc. Natl. Acad. Sci. USA 73: 1260-1264; Jaenisch et al. (1981) Cell 24: 519-529; Stuhlmann et al. (1984) Proc. Natl. Acad. Sci. USA 81: 7151-7159; Jahner et al. (1985) Proc. Natl. Acad. Sci. USA 82: 6927-6931; Van der Putten, et al. (1985) Proc. Natl. Acad Sci. USA 82: 6148-6152; Stewart, et al.(1987) EMBO J. 6: 383-388). In yet another approach based on viral infection, the retrovirus, or virus-producing cells, are injected into the blastocele (Jahner et al. (1982) Nature 298: 623-628). The foregoing methods have not gained wide acceptance because they all require laborious virus production and result in mosaicism.

Direct gene transfection into the adult germline has rarely been attempted due to difficulties in manipulating germ cells; limited success was reported with retrovirus-mediated gene delivery into male germ cells (Nagano (2000) FEBS Letters 475:7-10). Limitations of this approach arise from the lack of reliable culture methods that would permit long-term proliferation of germ cells, difficulties in gene integration, and unproven methods for selection in culture (Watanabe et al. (1997) Exp. Cell Res. 230: 76-83).

If successful, however, germline transfection would provide various important advantages over conventional methods. First, such direct modification of the germline would be the most efficient transgenesis method in some species by avoiding the tedious conventional procedures of embryo collection and transfer. Second, it would enable the study of gene function in germ cell development and gametogenesis immediately after transfection, even in cases when the transgene is known to cause embryonic lethality which eliminates production of fully transgenic offspring (Huang et al., supra). Finally, such direct methods may be the only ones that permit study of the integration of foreign vectors into chromosomes around the time of initiation of meiosis; the only period when endogenous homologous recombination occurs frequently along the chromosomes.

Chang and colleagues generated transgenic mice by lipofection of a transgene construct into mouse testes (Chang et al. (1999) J. Reprod. Dev. 45: 3742). This method, however, proved unreliable and inefficient. Furthermore, the transgenic mice failed to pass the transgene to sired progeny. See also Sato et al. (1999) Transgenics 3: 11-22.

The application of electroporation to mouse testes has so far met with limited success. Studies by Yamazaki and colleagues showed expression of marker genes two months after transfection in clusters of spermatogenic cells. However, very few spermatogenic cells were shown to carry, let alone express, the transgene. The study also failed to show expression of marker genes in mature spermatozoa (Yamazaki et al. (1998) Biol. Reprod. 59: 1439-1444), a prerequisite for their passage to progeny. The same investigators were unable to produce transgenic progeny mice sired by initial transfectants. In the latter study (Yamazaki et al. (2000) J. Exp. Zool. 286:212-218), the investigators successfully transferred a gene encoding green fluorescent protein (GFP), a well-known marker, in vivo to mouse spermatogenic cells. GFP was expressed in transfected testes not only in subsurface regions but also in the inner region of the testes indicating that the gene was transferred throughout the testis. GFP expression, however, remained transient. More importantly, there was no evidence of transgene transmission to offspring after mating. The total number of male germline cells with integrated transgene was low by histological analysis. The likely explanation was that the majority of sperm remained non-transgenic after electroporation and out-competed those sperm that were transgenic in the fertilization process.

The present invention is intended to solve these problems.

Spermatoenesis

Spermatogenesis is the three-step process by which stem cells develop into mature spermatozoa: spermatocytogenesis (mitosis), meiosis, and spermiogenesis. In the spermatocytogenesis stage, stem cells (Type A spermatogonia) divide mitotically to produce Type B spermatogonia that begin to differentiate by meiosis. During meiosis, cells in prophase of the first meiotic division form the primary spermatocytes, which are characterized by highly condensed chromosomes and intermediate position in the seminiferous epithelium. Primary spermatocytes proceed through the first meiotic division and become haploid secondary spermatocytes. The products of the second meiotic division are spermatids.

During spermiogenesis spherical spermatids metamorphose into elongated spermatozoa, the acrosome and the flagellar apparatus form, and most excess cytoplasm (the residual body) is separated and left in the Sertoli cell. Spermatozoa are released into the lumen of the seminiferous tubule. At all stages of differentiation, the spermatogenic cells are in close contact with Sertoli cells, which are thought to provide them with structural and metabolic support. Of particular interest to the present invention are the genetic factors that control spermatogenesis.

### SUMMARY OF THE INVENTION

The present invention provides a novel method of transgenesis and generation of transgenic mice that simplifies the laborious and time-consuming conventional pronucleus injection approach.

In a preferred embodiment of this method, a sterile male mouse deficient in spermatogenesis is transfected, preferably by electroporation, with a “transgenic construct” (which term is used to indicate a construct carrying a nucleic acid sequence to be incorporated into a host mammalian genome as a “transgene”). The transgenic construct also comprises a “spermatogenesis rescue cassette” (“SRC”).

As used herein, “spermatogenesis rescue cassette” (“SRC”) means a nucleic acid construct which, when expressed in a host deficient in spermatogenesis, overcomes that deficiency and restores normal or near-normal sperm production. A preferred SRC includes a nucleic acid sequence encoding a functional “spermatogenesis essential factor” (or “SEF”) which is a protein that is essential for spermatogenesis and, therefore, for normal or near normal male fertility. Known deleterious mutations in SEF genes lead to arrest of sperm production and sterility (Pittman, D L, et al. (1998) Mol. Cell. 1: 697-705).

The SEF in the preferred SRC is operably linked to a promoter that is operative in a mammal, preferably a mouse. A preferred promoter is that of the histone H1t gene (described by Bartell, J G et al., 1996, J. Biol. Chem. 271:4046-4054, for rat). The H1t gene is expressed exclusively in pachytene spermatocytes in mice (Drabent, B. et al., 1998, Cell Tissue Res 291:127-132).

In one embodiment, two loxP recombination sites flank the SRC in the transgenic construct. The loxP sites direct Cre recombinase-mediated excision of the SRC in transgenic progeny while the nucleic acid of interest remains incorporated in the genome.

Another embodiment utilizes the “Sleeping Beauty” (“SB”) Transposition system to augment integration of the gene of interest into the recipient genome. In one embodiment, the transgenic construct comprises an SRC optionally flanked by loxP recombination sites and a gene of interest, all of which is flanked by the nucleic acids sequence of the inverted repeated regions recognized by SB transposase. In this embodiment, sterile male mice deficient in spermatogenesis are co-transfected with a transgenic construct and a plasmid, preferably pCMV-SB, containing the SB transposase encoding gene. In another embodiment, such mice are transfected with the transgenic construct and mated to female mice transgenic for DNA encoding SB transposase (and Cre recombinase if the Cre/loxP recombination system is also used). In yet another embodiment DNA encoding the SB transposase is part of the construct comprising the gene of interest.

Specifically, the present invention is directed to a transgenic mouse whose germline cells comprise a transgene which includes: (a) a nucleic acid of interest, expressible in cells of the transgenic mouse, optionally operatively linked to a first promoter; and (b) an SRC comprising a nucleic acid encoding a SEF operatively linked to a second promoter, which SRC is optionally flanked at its 5′ and 3′ end by a loxP recombination site. In another embodiment, the transgenic mouse's cells comprise (a) a transgenic nucleic acid of interest, expressible in cells of the transgenic mouse, operatively linked to a first promoter; and (b) a single loxP recombination site either upstream or downstream from the nucleic acid. Preferred SEF's include Dmc1, Prp8 and Ccna1 (which encodes Cyclin A1).

In the above transgenic mouse the first promoter can, for example, be the hCMV promoter or a hEF promoter or any promoter operative in mice. The second promoter can also be any promoter operative in mice and is preferably the H1t promoter or a promoter natively linked to the SEF gene.

In another embodiment of the above transgenic mouse, the transgenic construct is flanked at its 5′ and 3′ ends by a nucleic acid sequence encoding SB transposon inverted repeats or partially functional mutants thereof. The nucleic acid of interest comprised within the transgenic construct flanked by the SB transposon, preferably becomes integrated in the genome in more than one location.

Also provided is a nucleic construct for introducing a transgene into a mammal comprising: (a) a nucleic acid of interest which is optionally operatively linked to a first promoter, (b) a SRC comprising a nucleic acid encoding a SEF operatively linked to a second promoter. In this construct the SRC may be flanked at its 5′ and 3′ end by a loxP recombination site. Preferred SEF's in the above construct include Dmc1, Prp8 and Ccna1. In this construct, the first or second promoter is an hCMV promoter or an hEF promoter. Another preferred second promoter is the H1t promoter, a promoter that is natively linked to the SEF gene. The nucleic acid of interest may be flanked at its 5′ and 3′ ends by a nucleic acid sequence encoding the SB transposon inverted repeats or flanked by a partially functional mutant thereof.

The above construct containing the SB inverted repeats may further comprise a nucleic acid sequence encoding a functional transposase (linked to a promoter) that recognizes the inverted repeats.

The present invention is also directed to a method of producing a transgenic mouse that expresses as a nucleic acid of interest, comprising the steps of:
(a) transfecting germ cells of a sterile spermatogenesis-deficient immature male mouse with the above which incorporates both the SRC and the nucleic acid of interest into the germ cells, thereby generating a transfectant mouse;
(b) permitting expression of the SEF in the transfectant mouse, thereby rescuing spermatogenesis;
(c) waiting a sufficient period for the transfectant mouse to mature sexually;
(d) mating the transfectant mouse to a female mouse to create F₁ progeny mice; and
(e) mating a resulting F₁ progeny mouse to a fertile mouse,
   thereby producing the transgenic mouse whose genome includes the nucleic acid of interest.

Another embodiment of this method comprises:
(a) transfecting germ cells of a sterile spermatogenesis-deficient immature male mouse with the above which incorporates both the SRC and the nucleic acid of interest into the germ cells, thereby generating a transfectant mouse;
(b) permitting expression of the SEF in the transfectant mouse, thereby rescuing spermatogenesis;
(c) waiting a sufficient period for the transfectant mouse to mature sexually,
(d) mating the transfectant mouse to a female mouse that is transgenic for DNA encoding Cre recombinase to create F₁ progeny mice, wherein, when the construct includes the lox P sites, the recombinase mediates excision of the SRC from the DNA of the F₁ progeny mice; and
(e) mating a resulting F₁ progeny mouse to a fertile mouse,
   thereby producing the transgenic mouse whose genome includes the transgenic nucleic acid of interest but not the SRC.

In one embodiment of the above method (i) the construct includes the loxP sites, and (ii) the female mouse of step (d) is transgenic for DNA encoding Cre recombinase, so that in the F₁ progeny mice, the recombinase mediates excision of the SRC from the DNA, thereby producing a transgenic mouse whose genome includes the transgenic nucleic acid of interest but not the SRC.

In the above method, transfecting is preferably accomplished by electroporation but may be accomplished by other means such as lipid mediated transfection, nucleic acid-coated microprojectile bombardment.

In a preferred embodiment of the above method, (a) in the construct, the nucleic acid of interest is flanked at its 5′ and 3′ ends by (i) nucleic acid sequences encoding SB transposon inverted repeats or (ii) partially functional mutants of the repeat-encoding sequences, and (b) the female mouse is also transgenic for DNA encoding (i) a transposase that recognizes the inverted repeats to which is linked a promoter. The method further comprises, after the mating step (d) above, the step of inducing transposase mediated transposition of the nucleic acid sequence of interest which thereby integrates in one or more sites of the genome of the F₁ progeny mouse.

Alternatively, a transgenic construct comprising the SB inverted repeats can be co-transfected along with a plasmid, preferably pCMV-SB, comprising DNA encoding the SB transposase.

### BRIEF DESCRIPTION OF THE DRAWINGS

FIG. 1 shows a schematic representation for generating transgenic mice using the present invention. The testes of a sterile, Dmc1^{-/-} male mouse are transfected with the transgenic construct shown in FIG. 2. The male transfected mouse matures and is mated to a female transgenic for Cre recombinase to produce F₁ progeny. The spermatogenesis rescue cassette (SRC) within the transgenic construct is excised in the F₁ progeny by way of Cre/loxP recombination. These F₁ progeny are then mated to wild type mice to yield progeny in which the Cre recombinase gene segregates out and the transgene-derived nucleic acid of interest is incorporated in the genome.

FIG. 2 shows an embodiment of the transgenic construct used in this invention. This transgenic construct comprises a SRC which includes a nucleic acid encoding a spermatogenesis essential factor (“SEF”) such as Dmc1, Prp8, or Ccna1 under the control of a promoter operative in mammalian, preferably murine, cells. The SRC is flanked by loxP sites. The transgenic construct also comprises a nucleic acid of interest (shown as “Gene X”) under the control of a second promoter operative in mammalian, preferably murine, cells.

FIG. 3 shows another embodiment of the transgenic construct used in this invention. This construct is similar to that depicted in FIG. 2 and further comprises 5′ and 3′ flanking nucleic acid sequences encoding the inverted repeats of the SB transposon.

FIG. 4 shows a schematic representation for generating transgenic mice in a similar way as shown in FIG. 1 using the transgenic construct depicted in FIG. 3.

The foregoing figures are provided for illustration purposes and in no way limit the scope of the claims.

### DESCRIPTION OF THE PREFERRED EMBODIMENTS

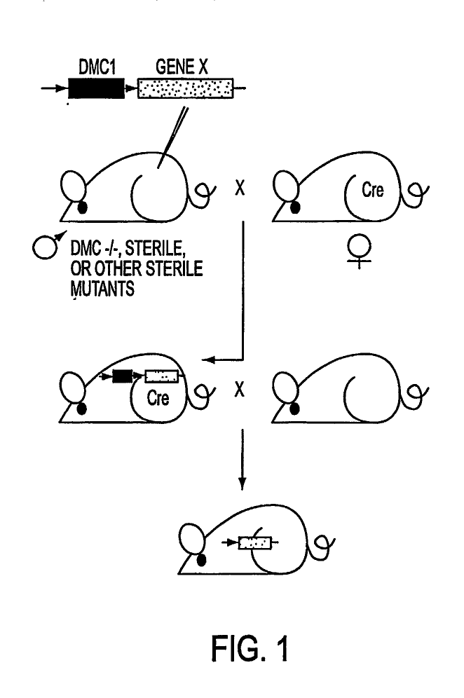

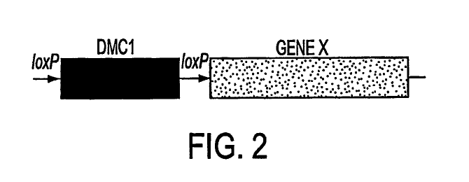

The key concept underlying this invention is the use of a male mouse that has been rendered sterile due to a mutation or mutations in a gene or genes encoding a “spermatogenesis essential factor” such as Dmc1, Prp8, or Ccna1. A transgenic construct such as that shown in FIG. 2 containing a wild type version of the mutant SEF gene is introduced into mouse testicular germ cells preferably by electroporation or other means. The expression of the wild type gene in the mutant germ cells restores normal spermatogenesis and promotes development of functional sperm. Since the mutant germ cells cannot produce functional sperm without expression of the SEF transgene, functional sperm that have been rescued by the transgenic construct also harbor the nucleic acid of interest that was part of that construct. When these transgenic male mice are later mated to normal females, their progeny will inherit the transgene (FIG. 1).

Transgenic Construct

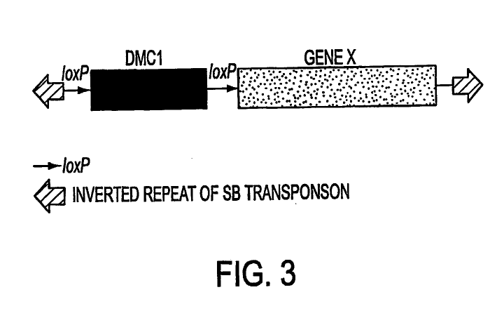

The transgenic construct, preferably linear, consists of two components as illustrated in FIGS. 2 and 3. The first component is the spermatogenesis rescue cassette, the SRC, having a wild type nucleic acid, preferably DNA, encoding an SEF used to rescue the mutant phenotype. Examples of the preferred SEF's are Dmc1, Prp8, and Ccna1.

The expression of this nucleic acid is preferably under the control of a germ cell-specific promoter, preferably, the H1t gene promoter (Kremer E J et al., 1992, Gene 110:167-173), although other, ubiquitous promoters can also be used.

Gene H1t encodes a testis-specific variant of the H1 histone family expressed in pachytene spermatocytes during the meiotic phase of spermatogenesis. In the initial description of this promoter, expression of the minimal promoter (174 nucleotides (nt) upstream from the transcription start point) was enhanced by sequences extending to nt-693, but was reduced in constructs with kb of upstream sequence. The H1t promoter is modulated both positively and negatively by distant upstream sequences in the native Ht1 gene structure.

In another embodiment, the promoter is an endogenous promoter of the gene encoding the SEF.

If desired, the SRC is flanked by loxP sites that will later facilitate excision of the rescue cassette, although such excision is optional. In fact, use of the CRE/loxP system as described herein is optional in the present methods.

The second component of the transgenic construct is the nucleic acid being inserted into the mouse's genome, i.e., the nucleic acid of interest, which is operatively linked to a separate promoter that is operative in mammalian, preferably murine, cells.

The promoter can be any one that drives expression of the nucleic acid of interest in mouse (male germline) cells. The promoter may be viral (e.g. hCMV promoter) or eukaryotic, preferably mammalian. Suitable promoters are inducible or repressible or, more preferably, constitutive. Preferred eukaryotic/viral promoters include the promoter of the mouse metallothionein I gene (Hamer, D., et al., J. Mol. Appl. Gen. 1:273-288 (1982)); the TK promoter of Herpes virus (McKnight, S., Cell 31:355-365 (1982)); the SV40 early promoter (Benoist, C., et al., Nature 290:304-310 (1981)); and the yeast gal4 gene promoter (Johnston, S. A., et al., Proc. Natl. Acad. Sci. (USA) 79:6971-6975 (1982); Silver, P. A., et al., Proc. Natl. Acad. Sci. (USA) 81:5951-5955 (1984)). A useful non-mammalian eukaryotic promoter is carp β-actin gene promoter (FV) (Ivics et al., Cell (1997) 91:501-510). Examples of vectors (plasmid or retrovirus) are disclosed in (Roy-Burman et al., U.S. Pat. No. 5,112,767). All of the above listed references are incorporated by reference in their entirety.

An example of a constitutive promoter is the viral promoter MSV-LTR, which is efficient and active in a variety of cell types, and, in contrast to most other promoters, has the same enhancing activity in arrested and growing cells. Other preferred viral promoters include that present in the hCMV-LTR (from cytomegalovirus) (Bashart, M. et al., Cell 41:521 (1985)) or in the RSV-LTR (from Rous sarcoma virus) (Gorman, C. M., Proc. Natl. Acad. Sci. USA 79:6777 (1982). Another preferred promoter is the human elongation factor 1α (“hEF”) promoter (Kim, D W et al., (1990) Gene 91:217-223).

Delivery of Transgenic Construct into Mouse Spermatogenic Cells

In a preferred embodiment, young male mice at postnatal day 14 are subjected to general anesthesia (e.g., with 2.5% avertin at a dose of 0.015-0.017 ml/g of body weight). Testes are pulled out with blunt forceps and allowed to rest on the fat pad. The transgenic construct in the form of a nucleic acid molecule preferably in a volume of 10-50 μl, is injected into the efferent ductules between testes and epididymis. In the electroporation embodiment described in more detail below, after injection the testes are held between a pair of electrodes (Tweezertrode, BTX) and 8 square 50 msec pulses at 10-50 V, are applied (BTX ECM 830 electroporator) to effect electroporation and uptake of the DNA. The testes are then reinserted into the body wall. The incision is closed with two to three stitches both for the body wall and the skin.

Electroporation

Electroporation, also known as electropermeabilization, is popularly used for transfection of cell suspensions. Forcep electrodes are used for in vivo electroporation aimed at various organs and tissue types (Muramatsu et al. (1998) J. Mol. Med. 1: 55-62). Electroporation entails exposing cells to short intense electric field pulses thereby inducing a transmembrane potential. The applied field induces temporary structural changes in the cell membrane, creating pathways from the extracellular space into the cell interior (Jaroszeski et al. (2000) Meth. Mol. Med. 37:173-186).

Currently, transmembrane induction voltages between 0.5 and 1 V minimum potential are used for most mammalian cell types. Induction potentials above this threshold can cause irreversible membrane damage and cell death (Teissié et al. (1992), In: Charge and Field Effects in Biosystems (Allen, M. J et al. eds.) Birkhauser, Boston, 3: 285-301); Teissié et al. (1982) Science 216:537-538).

Apart from the extent of membrane permeabilization, determined primarily through pulse duration and voltage, other factors control the intake of exogenous DNA by the cell. Most electroporation-mediated exogenous DNA transport is through electrophoresis rather than solely by membrane permeability (Klenchin et al. (1991) Biophys. J. 60:804-811, Wolf et al., Biophys. J. 66:524-531). Adding DNA immediately after the pulse usually results in lower transfection efficiency than when the DNA is added prior to the pulse. Shielding the charge of DNA by cations also reduces transfection efficiency (Anderson et al. (1989) 180: 269-275).

Most of the DNA entering the cell does so during the pulses by way of the electric field created across the membrane. As such, the transfection efficiency is proportional to the integral of the extent of membrane permeabilization with respect to the pulse time. For simple rectangular pulses or exponentially decaying pulses, the time integral is the pulse length T, which is usually much greater than the membrane relaxation time. If the permeabilized area of the cell is limited to polar regions, as is normally the case, the extent of membrane permeabilization (in terms of the number, density, and size of electropores) is approximately proportional to E-E_{b}, where E_{b} is the pulse field strength needed to produce the membrane breakdown voltage V_{b} (Hibino et al. (1991) Biophys. J 59: 209-220). Thus the transfection efficiency is roughly proportional to (E-E_{b})T (Hui et al. (2000) Meth. Mol. Med. 37: 157-171).

Nucleic Acid-Coated Microprojectile Bombardment

This method involves propulsion of nucleic acid-coated microprojectiles (preferably DNA) into target cells (Sanford et al. (1988) Particle Sci. Technol. 5: 27-37). Gold particles are particularly preferred.

A commercially available device (Biolistic PDS-1000; from Du Pont) uses a gunpowder discharge to impart momentum to coated projectiles. When performed in vitro, target cells are placed in a vacuum chamber during bombardment to minimize air impedance of particle flight.

Williams et al., (2000) Proc. Natl. Acad. Sci. USA 88:2726-2730, disclose a device for microprojectile bombardment to introduce and express exogenous nucleic acids directly in intact tissue of the living mouse. This device uses a helium discharge system, a disc macrocarrier for microprojectiles, and is configured to be hand-held. The use of helium gas permits more precise regulation of particle velocity and is configured such that the helium discharge impelling the microprojectiles is deflected away from the tissue, minimizing damage from the resulting shock wave. The helium discharge drives the macrocarrier through a 0.8 cm flight path to a stopping screen that arrests the macrocarrier disc but is permeable to nucleic acid coated microprojectiles which are permitted to strike the target tissue. Tissue becomes bombarded with coated gold particles (available from Alfa, Ward Hill, Mass.) having a range diameter between 1 and 3 μm or between 2 to 5 μm when tungsten particles (Sylvania) are used.

Microparticles are coated with nucleic acid by sequentially mixing gold or tungsten in an aqueous slurry comprising nucleic acid (about 1 mg/mL), CaCl₂ (2.5 M) and free-base spermidine (1 M). After 10 or so minutes of incubation, the microprojectiles are pelleted and the supernatant removed. The pellet is washed once with 70% ethanol, centrifuged, and resuspended in anhydrous ethanol. The nucleic acid coated microprojectiles are spread over the macrocarrier discs and allowed to dry in a dessicator before firing. The exact specifications of the device and its use are detailed in Williams, supra, which is hereby incorporated by reference.

A person having ordinary skill in the art will recognize that other approaches may be employed to introduce the transgenic construct into the germline such as viral infection or lipofection.

Lipid Mediated Transfection (Lipofection)

Some of the first work on liposome delivery of endogenous materials to cells occurred some twenty years ago. Foreign nucleic acids were introduced into cells using positively charged lipids. (Martin et al., (1976) J. Cell Biol. 70: 515-526, Magee et al., (1976) Biochim. Biophys. Acta 451: 610-618, and Straub et al., (1974) Infect. Immun. 10:783-792).

Of the many methods used to facilitate entry of DNA into eukaryotic cells, cationic liposomes are among the most efficacious and have found extensive use as DNA carriers in transfection experiments. (Thierry et al., Gene Regulation: Biology of Antisense RNA and DNA, p. 147 (Erickson and Izant, Eds., Raven Press, New York, 1992).

Senior et al., (1991) Biochim. Biophys. Acta 1070:173, suggested that incorporation of cationic lipids in liposomes is advantageous because it increases the amount of negatively charged molecules that can be associated with the liposome. In their study of the interaction between positively charged liposomes and blood, they concluded that harmful side-effects associated with macroscopic liposome-plasma aggregation can be avoided by limiting the dosage.

U.S. Pat. Nos. 5,695,780, 5,688,958, 5,686,620, 5,661,018, 5,651,981, herein incorporated by reference in their entirety, further elaborate the types of lipids useful in lipofection vectors and methodology used in the lipofection of nucleic acids into eukaryotic cells.

Spermatogenesis Essential Factors (SEF)

By combining transfection of mouse germ cells in the testes with an exogenous nucleic acid molecule (whether by electroporation, bombardment or other means), followed by integration, expression as a protein product in the germline, and passage of the incorporated nucleic acid to subsequent progeny, the present invention overcomes a limitation in the prior art (see Yamazaki et al., 2000, supra). A particular advantage of this invention over the art is the exploitation of spermatogenesis as a selection factor for enriching the stock of transgenic sperm available for fertilization.

The present invention exploits the genetic factors that control spermatogenesis. One such SEF is Dmc1, a meiosis specific gene first discovered in yeast that encodes a homologue of bacterial RecA and is implicated in recombination (Roca et al. (1990) Crit. Rev. Biochem. Mol. Biol. 25: 415-456). Yeast Dmc1 mutants and mutants of its murine homologue are defective in crossing over and synaptonemal complex formation and undergo arrest in late prophase meiosis I (Pittman et al., supra). Mammalian Dmc1 homologues have been isolated from mouse and human cDNA libraries (Sato et al. (1995) DNA Res. 2: 147-150); Habu et al. (1996) Nucleic Acid Res. 24: 470-477).

Both mouse and human Dmc1 genes encode a 340 amino acid protein containing the two nucleotide binding motifs (GEFRTGKT [SEQ ID NO: 1} and LLIID [SEQ ID NO: 2]) important for binding single and double-stranded DNA (Ogawa (1993) Cold Spring Harb. Symp. Quant. Biol. 58: 553-565). Transcription of mouse Dmc1 is restricted to testes and ovary, consistent with meiosis-specific expression in yeast
Dmc1 deficient mouse mutants show normal viability. However, crosses of Dmc1^{-/-} males to females failed to yield any births.

Furthermore, mouse Dmc1 mutants lack any mature spermatozoa. In normal mice, spermatogonial stem cells are found at the periphery of the seminiferous tubules. The seminiferous tubules from homozygous Dmc1^{-/-} mutants have arrested gamete development at the spermatocyte stage. Indeed, Dmc1^{-/-} testes are completely deficient in postmeoitic cells.

Another SEF that can be exploited in the present invention is the splicing factor Prp8. During mouse embryogenesis, Prp8 is expressed intensely at day 9.5 of gestation and its expression decreases progressively during embryogenesis. In adult mice, Prp8 is expressed strongly in the testes and moderately in the ovary. In situ hybridization analysis revealed that Prp8 is preferentially expressed in the outer cell layer in the testes (spermatogonia, primary spermatocytes, and in granulosa cells in the ovary). Unlike its yeast counterpart, which is essentially a U5 small nuclear RNP particle, vertebrate Prp8 has acquired an additional role in reproduction and spermatogenesis (Takahashi et al. (2001) J. Biochem. 129: 599-606).

Another SEF useful in the present invention is Cyclin A1 encoded by Ccna1. The mammalian A-type cyclin family consists of two members, cyclin A1 and A2. In mice, mutations in cyclin A2 are lethal while mutations in cyclin A1 produce viable but spermatogenesis-deficient sterile male mice. (Liu et al. (1998) Nature Genetics 20: 377-380). In Ccna1^{-/-} mice, approximately 10% of tubules contain spermatocytes undergoing apoptotic cell death, and the average testis weight is about 61% of that in wild-type mice. Moreover, the mutants failed to produce offspring when mated to normal females. Ccna1 is thus essential for spermatogenesis in early stage passage into the first meiotic division in male mice.

Cre/loxP Recombination System

One embodiment of this invention utilizes the Cre/loxP recombination system (see, e.g., U.S. Pat. No. 4,959,317) for excising the spermatogenesis rescue cassette from transgenic progeny. Cre recombinase of the P1 bacteriophage belongs to an integrase family of site-specific recombinases that is expressed in mammalian and other eukaryotic cell types (Saur et al. (1988) Proc. Natl. Acad. Sci. USA 85:5166-5170, (1989) Nuc. Acid. Res. 17:147-161, (1990) New Biol. 2:441-449). Cre recombinase is a 34 kDa protein that catalyzes recombination between two of its recognition sites called loxP. The loxP site is a 34 base pair consensus sequence consisting of a core spacer sequence of 8 base pairs and two flanking 13 base pair palindromic sequences.

One of the key advantages to this system is that there is no need for additional co-factors or sequence elements for efficient recombination regardless of cellular environment. Recombination occurs within the spacer area of the loxP sites. The post-recombination loxP sites are formed from the two complementary halves of the pre-recombination sites. The result of the Cre recombinase-mediated recombination depends on the location and orientation of the loxP sites. When an intervening sequence is flanked by similarly oriented loxP sites, as in the present invention, Cre recombinase activity results in excision. Cre/loxP recombination can be used at a high efficiency to excise a transgene in vivo (Orban et al. (1992) Proc. Natl. Acad. Sci. USA 89: 6861-6865).

Sleeping Beauty Transposon

In another embodiment, the transgenic construct incorporates the inverted repeats of the Sleeping Beauty (SB) transposon system such that activation of transposition by SB10 transposase integrates the gene of interest and/or the SRC; optionally flanked by loxp recombination sites, into various regions of the genome (Izsvak et al., J. Mol. Biol. (2000) 302:93-102).

The SB transposition system is a “cut-and-paste” type transposon member of the Tc1/mariner superfamily of salmonid transposable elements and is the only active DNA-based transposon system of vertebrate origin currently available for experimental manipulation.

Tc1/mariner transposons and transformation systems comprising them are described in U.S. Pat. Nos. 6,225,121, 6,159,717, 5,840,865, 5,348,874 to Savakis et al., and U.S. Pat. No. 6,051,430 to Plasterk et al., all of which are incorporated by reference in their entirety.

SB transposon functions in salmon, carp, mouse, and human cells. (Izsvak, supra.). Horie et al., Proc. Natl. Acad. Sci. USA. (2001) 98: 9191-9196, describes SB transposon-mediated creation of transgenic mouse lines expressing Enhanced Green Fluorescent Protein). There are two fundamental components of any mobile cut-and-paste transposition system: the first is an active transposase and the second is a DNA sequence recognized and mobilized by the transposase (Ivics et al., supra). SB has an inverted repeat/direct repeat (IR/DR) structure: directly repeated DNA sequence motifs at the ends of each approximately 230 bp imperfect inverted repeats. (Matches are less than 80% at the center of IRs; Ivics et al., supra.) The direct repeats (perfect repeats) are the core components of the binding sites for SB transposase. SB transposition requires the presence of two transposase binding sites within each inverted repeat. Mutant transposons having either insertions or deletions of any or all of any one binding site demonstrate a significant reduction in transposition efficiency. One mutant transposon that has one intact inverted repeat (having both transposase binding sites) and one partially deleted inverted repeat sequence (having only one functioning transposase binding site) demonstrated 26% transposition efficiency (Izsvak et al., supra). Therefore, mutants with partial function (defined herein as “partially functional mutants”) of SB are within the scope of this invention and preferably have at least about 20% of “normal” SB function. This permits a degree of control over transposition frequency and number of transposed DNA copies in transfectants.

One presently available SB transposase and encoding nucleic acid (SB10 transposase) is a synthetic construct bringing together known functional domains from public domain nucleic (Tss1.1 element from Atlantic Salmon, GenBank accession #L12206; and Tss1.2 element from Atlantic Salmon, accession #L12207, (Ivics et al., supra). This construct is available as plasmid, pCMV-SB. The nucleic acids including wild type and mutant SB inverted repeats are also available as plasmid, pT/MCS.

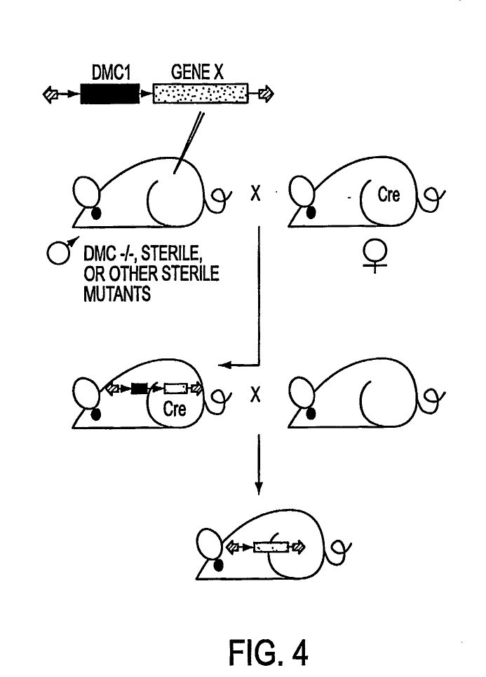

In one embodiment of the invention utilizing this transposition system, the germ cells of sterile males deficient in spermatogenesis are co-transfected with both a transgenic construct flanked by nucleic acids encoding the SB transposon inverted repeats (FIG. 3) and a plasmid comprising a DNA encoding the SB transposase (preferably pCMV-SB). SB transposase expression following transfection results in transposition of the transgenic construct and incorporation into multiple integration sites in the genome (FIG. 4).

In another embodiment employing this system, SB transposase encoding DNA with an operatively linked promoter are incorporated into the transfected transgenic construct.

Genetic Crosses

The general breeding scheme employed to carry out genetic crosses that produce transgenic progeny is illustrated in FIG. 1.

After electroporation or other form of DNA uptake, the presumptively transgenic mouse is allowed to recover from the surgery and to mature. When sexually mature, it is mated to a female mouse. If the Cre/loxP system is used, the female is transgenic for Cre recombinase encoding nucleic acid under the control of hCMV promoter.

If the SB transposition system is used, it can be activated either by co-transfection; as described above, or by mating the male transfected with the transgenic construct with a female that is transgenic for the SB transposase which mediates transposition of the region flanked by the SB inverted repeat into other locations of the transgenic mouse genome.

The F₁ progeny of the above types of crosses are genotyped to ensure that they have the relevant transgenes in their respective genomes.

The SEFs will be excised from the germline in the F₂ generation at the time that Cre recombinase acts at the loxP recombination sites of progeny of the F₁ mating.

The hCMV-Cre transgene segregates out by crossing these F₁ progeny to a wild type strain to yield an F₂ generation having only the transgene derived nucleic acid of interest flanked by a single upstream loxP site.

The references cited above are all incorporated by reference herein, whether specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

1. A nucleic acid construct for introducing a transgene into a mammal comprising
(a) a nucleic acid of interest which is optionally operatively linked to a first promoter,
(b) a spermatogenesis rescue cassette comprising a nucleic acid encoding a spermatogenesis essential factor (SEF) operatively linked to a second promoter.

2. The construct of claim 1 wherein the spermatogenesis rescue cassette is flanked at its 5′ and 3′ end by a loxP recombination site.

3. he construct of claim 1 wherein the SEF is selected from the group consisting of Dmc1, Prp8 and Ccna1.

4-5. canceled

6. The construct of claim 1 wherein the first promoter is an hCMV promoter or an hEF promoter and the second promoter is (a) an hCMV promoter (b) an hEF promoter; (c) the H1t promoter or (d) a promoter natively linked to a SEF gene encoding said SEF.

7-8. canceled

9. The construct of claim 1 further comprising at its 5′ and 3′ ends, (i) nucleic acid sequences encoding SB transposon inverted repeats or (ii) a partially functional mutant of said repeat-encoding sequence.

10. The construct of claim 6 further comprising at its 5′ and 3′ ends, (i) nucleic acid sequences encoding SB transposon inverted repeats or (ii) a partially functional mutant of said repeat-encoding sequence.

11-12. canceled

13. The construct of claim 9 further comprising a nucleic acid sequence encoding a functional transposase that recognizes said inverted repeats and a promoter operatively linked thereto.

14. The construct of claim 10 further comprising a nucleic acid sequence encoding a functional transposase that recognizes said inverted repeats and a promoter operatively linked thereto.

15-16. canceled

17. A method of producing a transgenic mouse that expresses a nucleic acid of interest, comprising the steps of:
(a) transfecting germ cells of a sterile spermatogenesis-deficient immature male mouse with the construct of claim 1 which incorporates both said spermatogenesis rescue cassette and said nucleic acid of interest into said germ cells, thereby generating a transfectant mouse;
(b) permitting expression of said SEF in said transfectant mouse, thereby rescuing spermatogenesis;
(c) waiting a sufficient period for said transfectant mouse to mature sexually;
(d) mating the transfectant mouse to a female mouse to create F₁ progeny mice; and
(e) mating a resulting F₁ progeny mouse to a fertile mouse,
thereby producing said transgenic mouse whose genome includes said nucleic acid of interest.

18. A method of producing a transgenic mouse that expresses a nucleic acid of interest, comprising the steps of:
(a) transfecting germ cells of a sterile spermatogenesis-deficient immature male mouse with the construct of claim 6 which incorporates both said spermatogenesis rescue cassette and said nucleic acid of interest into said germ cells, thereby generating a transfectant mouse;
(b) permitting expression of said SEF in said transfectant mouse, thereby rescuing spermatogenesis;
(c) waiting a sufficient period for said transfectant mouse to mature sexually;
(d) mating the transfectant mouse to a female mouse to create F₁ progeny mice; and
(e) mating a resulting F₁ progeny mouse to a fertile mouse,
thereby producing said transgenic mouse whose genome includes said nucleic acid of interest.

19-20. canceled

21. A method of producing a transgenic mouse that expresses a nucleic acid of interest, comprising the steps of:
(a) transfecting germ cells of a sterile spermatogenesis-deficient immature male mouse with the construct of claim 9 which incorporates both said spermatogenesis rescue cassette and said nucleic acid of interest into said germ cells, thereby generating a transfectant mouse;
(b) permitting expression of said SEF in said transfectant mouse, thereby rescuing spermatogenesis;
(c) waiting a sufficient period for said transfectant mouse to mature sexually;
(d) mating the transfectant mouse to a female mouse to create F₁ progeny mice; and
(e) mating a resulting F₁ progeny mouse to a fertile mouse,
thereby producing said transgenic mouse whose genome includes said nucleic acid of interest.

22. A method of producing a transgenic mouse comprising:
(a) transfecting germ cells of a sterile spermatogenesis-deficient male mouse with the construct of claim 1 which incorporates both said spermatogenesis rescue cassette and said nucleic acid of interest into said germ cells, thereby generating a transfectant mouse;
(b) permitting expression of said SEF in said transfectant mouse, thereby rescuing spermatogenesis;
(c) waiting a sufficient period for said transfectant mouse to mature sexually;
(d) mating the transfectant mouse to a female mouse that is transgenic for DNA encoding Cre recombinase to create F₁ progeny mice, wherein, when said construct includes said loxP sites, said Cre recombinase mediates excision of said spermatogenesis rescue cassette from the DNA of said F₁ progeny mice; and
(e) mating a resulting F₁ progeny mouse to a fertile mouse,
thereby producing said transgenic mouse whose genome includes said transgenic nucleic acid of interest but not said spermatogenesis rescue cassette.

23. A method of producing a transgenic mouse comprising:
(a) transfecting germ cells of a sterile spermatogenesis-deficient male mouse with the construct of claim 6 which incorporates both said spermatogenesis rescue cassette and said nucleic acid of interest into said germ cells, thereby generating a transfectant mouse;
(b) permitting expression of said SEF in said transfectant mouse, thereby rescuing spermatogenesis;
(c) waiting a sufficient period for said transfectant mouse to mature sexually;
(d) mating the transfectant mouse to a female mouse that is transgenic for DNA encoding Cre recombinase to create F₁ progeny mice, wherein, when said construct includes said loxP sites, said Cre recombinase mediates excision of said spermatogenesis rescue cassette from the DNA of said F₁ progeny mice; and
(e) mating a resulting F₁ progeny mouse to a fertile mouse,
thereby producing said transgenic mouse whose genome includes said transgenic nucleic acid of interest but not said spermatogenesis rescue cassette.

24-25. canceled

26. A method of producing a transgenic mouse comprising:
(a) transfecting germ cells of a sterile spermatogenesis-deficient male mouse with the construct of any of claim 9 which incorporates both said spermatogenesis rescue cassette and said nucleic acid of interest into said germ cells, thereby generating a transfectant mouse;
(b) permitting expression of said SEF in said transfectant mouse, thereby rescuing spermatogenesis;
(c) waiting a sufficient period for said transfectant mouse to mature sexually;
(d) mating the transfectant mouse to a female mouse that is transgenic for DNA encoding Cre recombinase to create F₁ progeny mice, wherein, when said construct includes said loxP sites, said Cre recombinase mediates excision of said spermatogenesis rescue cassette from the DNA of said F₁ progeny mice; and
(e) mating a resulting F₁ progeny mouse to a fertile mouse,
thereby producing said transgenic mouse whose genome includes said transgenic nucleic acid of interest but not said spermatogenesis rescue cassette.

27. The method of claim 17 wherein
(i) said construct includes said loxP sites,
(ii) said female mouse of step (d) is transgenic for DNA encoding Cre recombinase, so that in said F₁ progeny mice, said recombinase mediates excision of said spermatogenesis rescue cassette from the DNA,
thereby producing a transgenic mouse whose genome includes said transgenic nucleic acid of interest but not said spermatogenesis rescue cassette.

28. The method of claim 18 wherein
(i) said construct includes said loxP sites,
(ii) said female mouse of step (d) is transgenic for DNA encoding Cre recombinase, so that in said F₁ progeny mice, said recombinase mediates excision of said spermatogenesis rescue cassette from the DNA,
thereby producing a transgenic mouse whose genome includes said transgenic nucleic acid of interest but not said spermatogenesis rescue cassette.

29-30. canceled

31. The method of claim 21 wherein
(i) said construct includes said loxP sites,
(ii) said female mouse of step (d) is transgenic for DNA encoding Cre recombinase, so that in said F₁ progeny mice, said recombinase mediates excision of said spermatogenesis rescue cassette from the DNA,
thereby producing a transgenic mouse whose genome includes said transgenic nucleic acid of interest but not said spermatogenesis rescue cassette.

32. The method of claim 22 wherein
(i) said construct includes said loxP sites,
(ii) said female mouse of step (d) is transgenic for DNA encoding Cre recombinase, so that in said F₁ progeny mice, said recombinase mediates excision of said spermatogenesis rescue cassette from the DNA,
thereby producing a transgenic mouse whose genome includes said transgenic nucleic acid of interest but not said spermatogenesis rescue cassette.

33. The method of claim 23 wherein
(i) said construct includes said loxP sites,
(ii) said female mouse of step (d) is transgenic for DNA encoding Cre recombinase, so that in said F₁ progeny mice, said recombinase mediates excision of said spermatogenesis rescue cassette from the DNA,
thereby producing a transgenic mouse whose genome includes said transgenic nucleic acid of interest but not said spermatogenesis rescue cassette.

34-35. canceled

36. The method of claim 26 wherein
(i) said construct includes said loxP sites,
(ii) said female mouse of step (d) is transgenic for DNA encoding Cre recombinase, so that in said F₁ progeny mice, said recombinase mediates excision of said spermatogenesis rescue cassette from the DNA,
thereby producing a transgenic mouse whose genome includes said transgenic nucleic acid of interest but not said spermatogenesis rescue cassette.

37. The method of claim 17 wherein the transfecting is by electroporation, nucleic acid coated microprojectile bombardment or lipid mediated transfection.

38-39. canceled

40. The method of claim 22 wherein the transfecting is by electroporation, nucleic acid coated microprojectile bombardment or lipid mediated transfection.

41-45. canceled

46. The method of claim 32 wherein the transfecting is by electroporation, nucleic acid coated microprojectile bombardment or lipid mediated transfection.

47-48. canceled

49. The method of claim 17 wherein the transfecting step further comprises co-transfecting said germ cells of said sterile spermatogenesis-deficient male mouse with a plasmid comprising DNA encoding an SB transposase,
which method further comprises, after transfection, the step of expressing said SB transposase such that transposon mediated integration incorporates the construct in one or more sites of the transfected mouse genome.

50-53. canceled

54. The method of claim 22 wherein the transfecting step further comprises co-transfecting said germ cells of said sterile spermatogenesis-deficient male mouse with a plasmid comprising DNA encoding an SB transposase,
which method further comprises, after transfection, the step of expressing said SB transposase such that transposon mediated integration incorporates the construct in one or more sites of the transfected mouse genome.

55-69. canceled

70. The method of claim 17 wherein the construct used in the transfecting step further comprises DNA encoding an SB transposase and an operatively linked promoter, said method further comprising, after transfection, the step of expressing said SB transposase such that transposon mediated integration incorporates the construct in one or more sites of the transfected mouse genome.

71. The method of claim 22 wherein the construct used in the transfecting step further comprises DNA encoding an SB transposase and an operatively linked promoter, said method further comprising, after transfection, the step of expressing said SB transposase such that transposon mediated integration incorporates the construct in one or more sites of the transfected mouse genome.

72. canceled

73. The method of claim 32 wherein the construct used in the transfecting step further comprises DNA encoding an SB transposase and an operatively linked promoter, said method further comprising, after transfection, the step of expressing said SB transposase such that transposon mediated integration incorporates the construct in one or more sites of the transfected mouse genome.

74. The method of claim 17 wherein the female mouse of the mating step is further transgenic for DNA encoding SB transposase, and further comprising after mating the step of expressing said SB transposase such that transposon mediated integration incorporates the construct in one or more sites of the transfected mouse genome.

75. The method of claim 22 wherein the female mouse of the mating step is further transgenic for DNA encoding SB transposase, and further comprising after mating the step of expressing said SB transposase such that transposon mediated integration incorporates the construct in one or more sites of the transfected mouse genome.

76. The method of claim 27 wherein the female mouse of the mating step is further transgenic for DNA encoding SB transposase, and further comprising after mating the step of expressing said SB transposase such that transposon mediated integration incorporates the construct in one or more sites of the transfected mouse genome.

77. The method of claim 32 wherein the female mouse of the mating step is further transgenic for DNA encoding SB transposase, and further comprising after mating the step of expressing said SB transposase such that transposon mediated integration incorporates the construct in one or more sites of the transfected mouse genome.

78. A transgenic mouse whose germline cells comprise a transgene which includes:
(i) a nucleic acid of interest, operatively linked to a first promoter; and expressible in cells of said transgenic mouse; and
(ii) a spermatogenesis rescue cassette comprising a nucleic acid encoding a SEF operatively linked to a second promoter, which cassette is optionally flanked at its 5′ and 3′ end by a loxP recombination site.

79. A transgenic mouse whose cells comprise:
(i) a transgenic nucleic acid of interest, operatively linked to a first promoter; and expressible in cells of said transgenic mouse; and
(ii) a single loxP recombination site either upstream or downstream from said nucleic acid of interest.

80. The transgenic mouse of claim 78 wherein the SEF is Dmc1, Prp or Ccna1.

81-82. canceled

83. The transgenic mouse of claim 78 wherein the first promoter is an hCMV promoter or a hEF promoter and the second promoter is the H1t promoter or a promoter natively linked to the SEF gene.

84-88. canceled

89. The transgenic mouse of claim 79 wherein the first promoter is an hCMV promoter or a hEF promoter and the second promoter is the H1t promoter or a promoter natively linked to the SEF gene.

90-93. canceled

94. The transgenic mouse of claim 78 further comprising (i) nucleic acid sequences encoding SB transposon inverted repeats or (ii) a partially functional mutant of said repeat-encoding sequence.

95. The transgenic mouse of claim 79 further comprising (i) nucleic acid sequences encoding SB transposon inverted repeats or (ii) a partially functional mutant of said repeat-encoding sequence.

96. The transgenic mouse of claim 80 further comprising (i) nucleic acid sequences encoding SB transposon inverted repeats or (ii) a partially functional mutant of said repeat-encoding sequence.

97-98. canceled

99. The transgenic mouse of claim 83 further comprising (i) nucleic acid sequences encoding SB transposon inverted repeats or (ii) a partially functional mutant of said repeat-encoding sequence.
